(19) United States
(12) Patent Application Publication
(10) Pub. No.: US 2025/0375258 A1
(43) Pub. Date: Dec. 11, 2025

(54) ENCLOSURE FOR MITIGATION OF LEAKAGE BY SYRINGE ASSEMBLY

(71) Applicant: Merck Sharp & Dohme LLC, Rahway, NJ (US)

(72) Inventors: Derrick Matthew Smith, Newtown Square, PA (US); Christopher Hsiu-Cheng Lee, Cary, NC (US)

(21) Appl. No.: 18/758,424

(22) Filed: Jun. 28, 2024

Related U.S. Application Data
(63) Continuation-in-part of application No. 18/740,352, filed on Jun. 11, 2024.

(51) Int. Cl. A61B 90/00; A61M 5/32

(52) U.S. Cl. CPC A61B 90/05; A61M 5/3216

(57) ABSTRACT

A splash guard engages to a syringe assembly to mitigate splashing of liquid in instances of failure of the syringe assembly. The splash guard forms an enclosure around the syringe assembly, such that, in the event of failure, the liquid or an aerosol form of the liquid is contained within the enclosure. The splash guard may include a handle for engaging and disengaging the splash guard from the syringe assembly. As a user applies an actuation force on the handle, the splash guard opens into an opened state. In the opened state, the user can position the splash guard around the syringe assembly before releasing the handle and returning to the splash guard to a closed state.

### CROSS REFERENCE TO RELATED APPLICATION

This application is a continuation-in-part of U.S. Patent Application No.: 18/740,352, filed Jun. 11, 2024, which is incorporated by reference.

### BACKGROUND

Syringe filtration is an important tool in a wet lab setting. The syringe assembly typically includes a syringe coupled to a filter.

The syringe assembly can be prone to failure from built up pressure in the filter, causing the filter to dissemble and splashing the liquid. Such liquid may be hazardous, creating a safety hazard in unintentional leakage or aerosolization of the liquid.

### SUMMARY

A splash guard contains leakage by a syringe assembly. The splash guard includes two enclosure halves. Each enclosure half comprises a body formed from a side wall, a top wall connected to the side wall along a top edge of the side wall, a bottom wall connected to the side wall along a bottom edge of the side wall, and an internal protrusion connected to an inner surface of the side wall and structured to guide the syringe assembly to a particular placement within the enclosure. Each enclosure half further comprises an arm comprising a first end connected to an outer surface of the side wall opposite the inner surface, and a second end opposite the first end for gripping by a user. The two enclosure halves are pivotably coupled with a hinge biasing the arms towards an equilibrium state in which the second ends of the arms are positioned away from one another and the first ends of the arms impel the two enclosure halves together such that the two enclosure halves form a substantially enclosed cavity when engaged with the syringe assembly. When engaged to the syringe assembly, the splash guard mitigates splashing of the fluid due to failure of the syringe assembly.

### BRIEF DESCRIPTION OF THE DRAWINGS

FIG. 1A illustrates a perspective view of the splash guard for mitigation of leakage by a syringe assembly in an equilibrium state, according to one or more embodiments.

FIG. 1B illustrates a perspective view of the splash guard of FIG. 1A in an actuated state, according to one or more embodiments.

FIG. 2A illustrates an internal view of an enclosure half of the splash guard, according to one or more embodiments.

FIG. 2B illustrates a perspective view of the enclosure half of the splash guard, according to one or more embodiments.

FIG. 3A illustrates a first view of the splash guard disengaged from a syringe assembly, according to one or more embodiments.

FIG. 3B illustrates a second view of the splash guard engaged to a syringe assembly, according to one or more embodiments.

FIG. 4 illustrates an example 3D printer assembly, according to one or more embodiments.

FIG. 5 illustrates a method flowchart for a process of manufacturing a splash guard for mitigating leakage by a syringe assembly, according to one or more embodiments.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### Overview

A splash guard engages to a syringe assembly including a syringe and a filter to mitigate splashing of liquid in instances of failure of the syringe assembly. In one or more embodiments, the splash guard forms an enclosure around the syringe assembly, such that, in the event of failure, the liquidd or an aerosol form of the liquid is contained within the enclosure. The splash guard may include a handle for engaging and disengaging the splash guard from the syringe assembly. The handle may be formed from two arms attached to the enclosure. In a closed state, the splash guard remains closed. As a user applies an actuation force on the handle, the splash guard opens into an opened state. In the opened state, the user can position the splash guard around the syringe assembly before releasing the handle and returning to the splash guard to the closed state.

Failure of the syringe assembly include failure of the syringe, failure of the filter, failure of a connection between the syringe and the filter, etc. For example, poor construction of the syringe may cause one or more portions of the syringe to break the waterproof seal, causing leakage of the liquid held in the syringe. As another example, failure of the syringe and/or the filter may include disassembly of components of the syringe and/or the filter, also leading to leakage of the liquid from the syringe assembly. In a third example, the filter may be loosely connected to the syringe. In such instances, built up pressure from the filter can cause the filter to disconnect (wholly or partially) from the syringe, thereby causing leakage. In such failures, the splash guard mitigates splashing of the liquid by containing the leaked liquid in the enclosure.

### Splash Guard

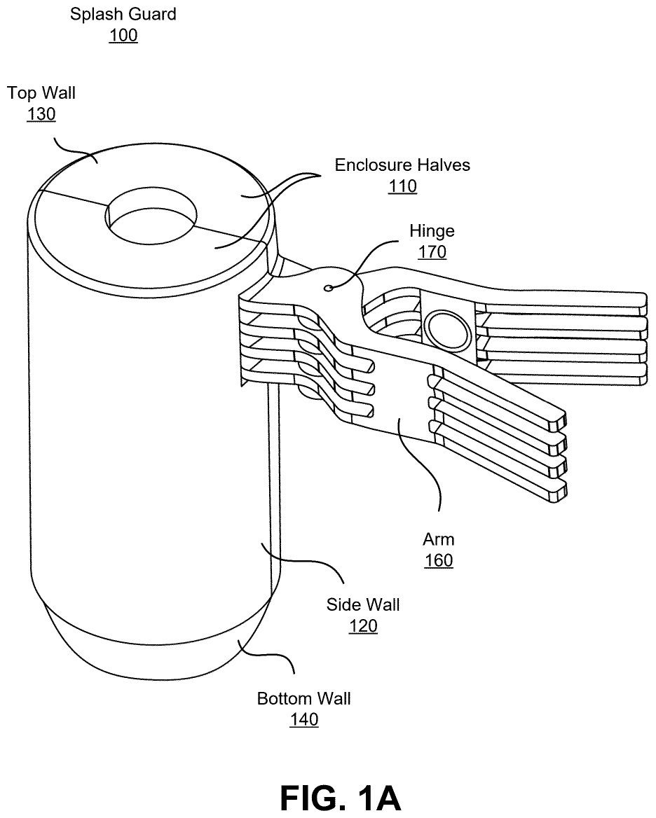

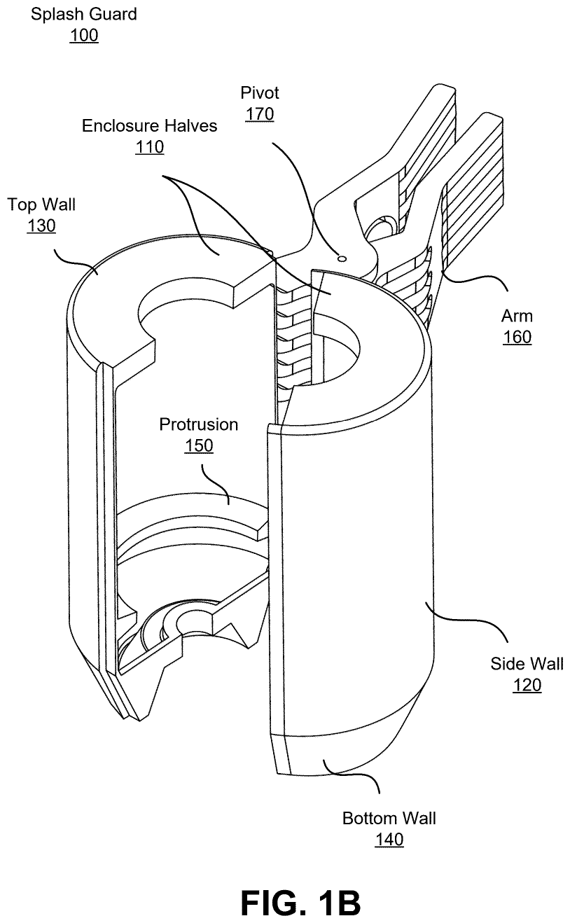

FIG. 1A illustrates a perspective view of the splash guard 100 for mitigation of leakage by a syringe assembly in a closed state, according to one or more embodiments. FIG. 1B illustrates a perspective view of the splash guard 100 of FIG. 1A in an opened state, according to one or more embodiments. The splash guard 100 comprises two enclosure halves 110 that are pivotably coupled together with a hinge 170. Each enclosure half 110 includes a side wall 120, a top wall 130, a bottom wall 140, and an arm 160. In some embodiments, each enclosure half 110 further includes a protrusion 150 positioned within the enclosure.

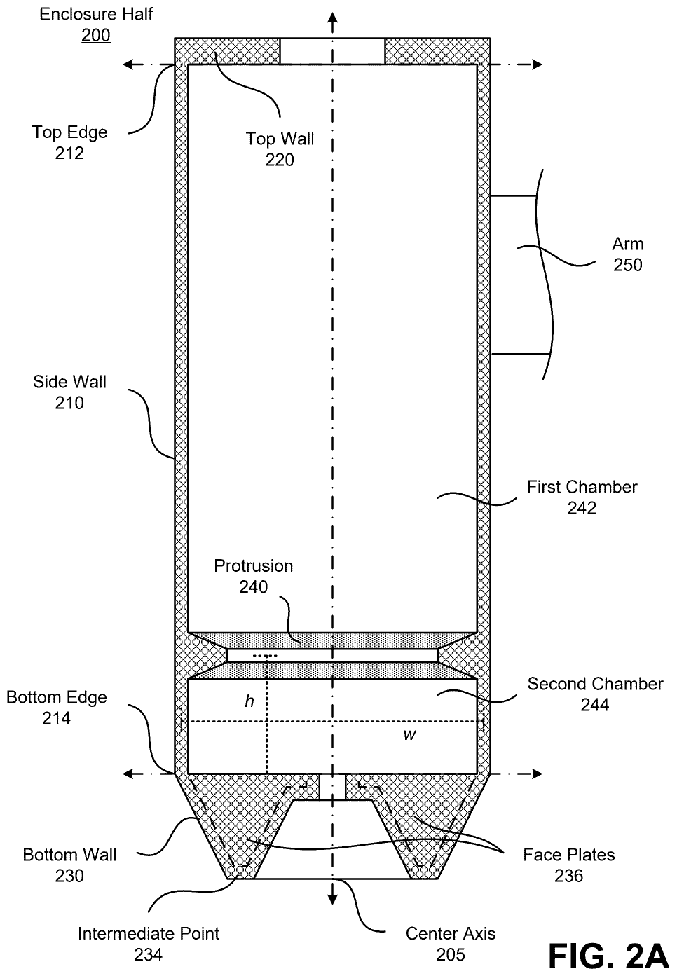

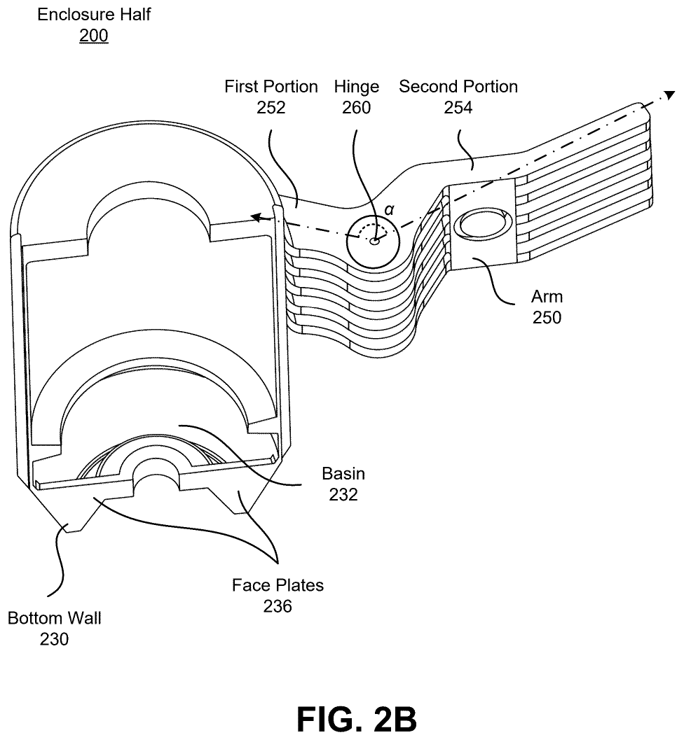

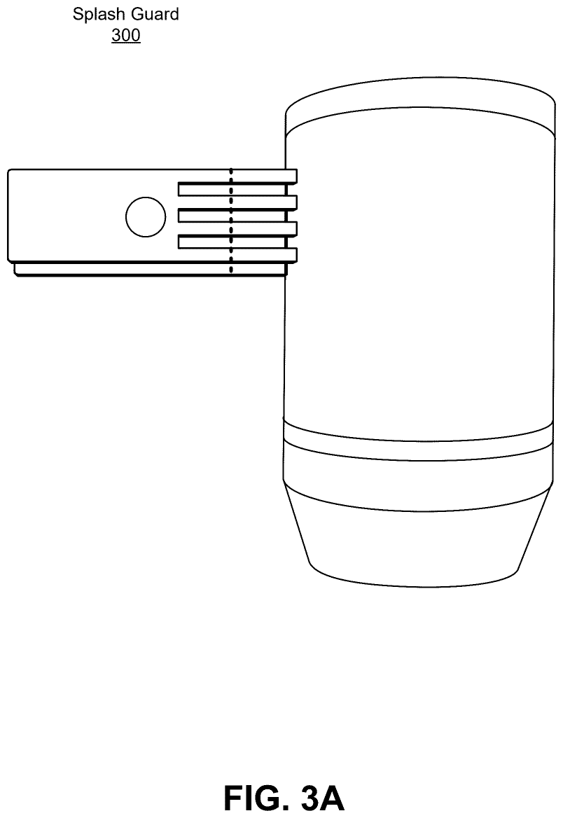

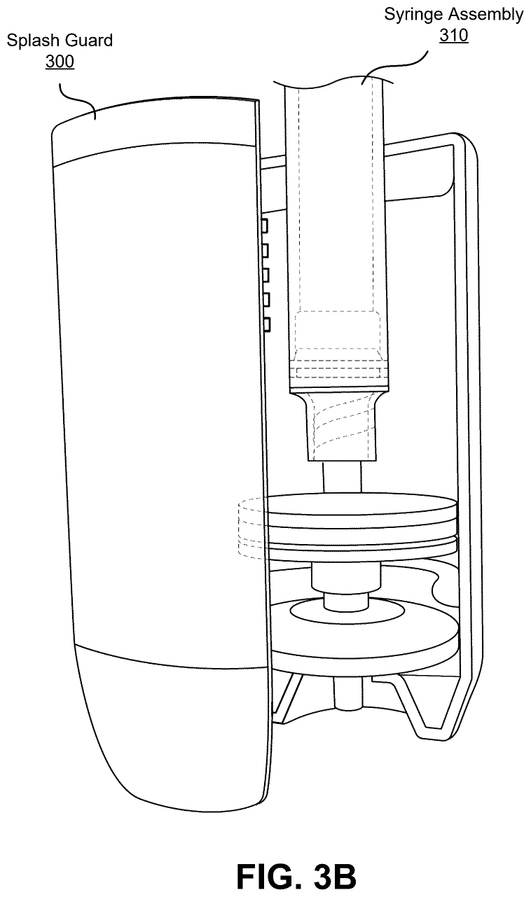

The enclosure is a cavity formed by the side walls 120, the top walls 130, and the bottom walls 140 of the enclosure halves 110. The side walls 120 run lengthwise with the syringe assembly, along an axis where a plunger of the syringe translates about a barrel of the syringe (an example of which is shown in FIG. 3B). The side walls 120 may be sized to fit at least the portion of the syringe assembly including the filter. The side wall 120 of one enclosure half 110 may include a top edge and a bottom edge opposite the top edge. Further, the side wall 120 may include a first longitudinal edge and a second longitudinal edge opposite the first longitudinal edge. The top wall 130 of one enclosure half 110 connects to a top edge of the side wall 120. The top walls 130 may include a cutaway portion such that a top portion (e.g., the syringe or the barrel of the syringe) of the syringe assembly can extend out of a top of the splash guard 100. When the top walls 130 of the two enclosure halves 110 are in contact, i.e., in the closed state of the syringe guard, the cutaway portion may be sized to fit the syringe of the syringe assembly. This can contain any splashes directed toward the top of the enclosure. The bottom wall 140 of one enclosure half 110 connects to a bottom edge of the side wall 120. The bottom walls 140 also include a cutaway portion such that a bottom portion (e.g., the filter or an orifice of the filter) of the syringe assembly can extend out of a bottom of the splash guard 100 (e.g., an orifice of the filter). The bottom walls 140 prevent leakage out of the bottom of the enclosure. Additional details relating to the enclosure halves 110 is disclosed in conjunction with FIGS. 2A & 2B.

In one or more embodiments, the enclosure further includes a protrusion 150 per enclosure half 110 positioned internally (shown in FIG. 1B). The protrusion 150 is connected to an inner surface of the side wall 120. The protrusion 150 may be positioned closer to the bottom edge of the side wall 120 and further from the top edge. The protrusion 150 can aid in positioning the splash guard 100 in an optimal placement relative to the syringe assembly. In particular, the protrusion aids in vertical alignment of the syringe assembly such that the bottom portion of the syringe assembly extends out of the bottom orifice of the enclosure. In one or more embodiments, the protrusions 150 and the bottom walls 140 secure a top and a bottom of the filter of the syringe assembly (or a bottom filter of a plurality of filters of the syringe assembly), respectively.

In one or more embodiments, the enclosure may have a cylindrical shape. Each enclosure half 110 (or at least some portion thereof) is of a semi-cylindrical shape. Accordingly, the side wall 120 of one enclosure half 110 is of curved shape corresponding to the circumference of the semi-cylinder shape. The top wall 130 of one enclosure half 110 may be of semi-annular shape including the cutaway portion for the syringe assembly extending out of the top of the splash guard 100. The cutaway portion corresponds to an inner radius of the annular shape. The top wall 130 may include an outer semi-circumferential edge that couples to the top edge of the side wall 120 and an inner semi-circumferential edge forming the cutaway portion. The top wall 130 may further include two radial edges connecting the outer semi-circumferential edge and the inner semi-circumferential edge. The bottom wall 140 of one enclosure half 110 may also be of semi-annular shape including the cutaway portion for the syringe assembly extending out of the bottom of the splash guard 100. In one or more embodiments, the top walls 130 and/or the bottom walls 140 of the enclosure halves 110 are substantially planar and perpendicular to a center longitudinal axis of the cylindrical enclosure. Each protrusion 150 may also have semi-annular shape, substantially planar, perpendicular to the center longitudinal axis, or some combination thereof.

In one or more embodiments, one enclosure half 110 may include an overhang edge that couples to an indented edge of the other enclosure half 110. The overhang edge and the indented edge may extend along one or more edges of the enclosure half. For example, as shown in FIG. 1B, the overhang edge extends along the edges of the side wall 120 on the right enclosure half 110. The indented edge extends along the edges of the side wall 120 on the left enclosure half 110. When the two enclosure halves are in the closed state, the overhang edge keys into the indented edge, such that the overhang edge crosses the bifurcation plane separating the two enclosure halves 110. In additional embodiments, there can be an additional overhang edge, e.g., along the top wall 130 and/or the bottom wall 140.

In one or more embodiments, the enclosure is formed of a material that is at least partially transparent. The partial (or full) transparency permits a user to visually assess the syringe assembly when the splash guard 100 is engaged to the syringe assembly. In another example, the user can assess whether liquid has leaked out of the syringe assembly and has been contained by the splash guard, e.g., due to failure of the syringe assembly.

In one or more embodiments, the splash guard 100 may include one or more seals coupled to one or more edges of the enclosure. The seals may help to provide a waterproof seal between one or more edges. For example, one seal may be positioned along an inner edge of the top wall 130 to form a seal between the top wall 130 and the syringe assembly. As another example, a seal may be positioned along an inner edge of the bottom wall 140 to form a seal between the bottom wall 140 and the syringe assembly. In yet another example, a seal may be positioned along an inner edge of one of the bottom walls 140 to form a seal with the other bottom wall 140.

The arms 160 of the enclosure halves 110 form a handle for actuating the splash guard 100. Each arm 160 may include a first end coupled to an external surface of the side wall 120, that is opposite an inner surface. Each arm 160 may extend away from the side wall 120, e.g., in a perpendicular direction to the outer surface. Each arm 160 may further include a second end opposite the first end, where a user can grip the splash guard 100. Each arm 160 may include a hole for establishing a hinge 170 between the two enclosure halves 110. The arms may include counterpart comb-like cutaway portions around the hole that interlock the cutaway portions of the two arms 160 together. As shown in FIG. 1B, at a first end of each arm 160, the arm 160 includes three cutaway portions forming four digits at the first end. One end of the four digits connects the arm 160 to the enclosure, while the other end of the digits extends to an intermediate point of the arms 160. In an intermediate point of the digits (i.e., the comb-like cutaway portion), the digits of each arm 160 interlock at the hinge 170. A rod may be inserted into the holes of the arms 160 to join the enclosure halves 110 together, thereby forming the hinge. The arms 160 may be angled at the hinge 170 (i.e., in a plane perpendicular to the longitudinal axis of the enclosure). The arms 160 are angled such that the hinge 170 is disposed along the bifurcation plane between the two enclosure halves 110. In one or more embodiments, a spring further joins the arms 160 together, to impel the enclosure into the closed state. In the closed state (i.e., in equilibrium), the second ends of the arms 170 are biased away from one another. In some embodiments, the spring is a torsion spring. To open the enclosure, a user applies an actuation force to draw the second ends of the arms 170 towards one another, thereby causing the enclosure to hinge into the opened state.

In one or more embodiments, the splash guard 100 is formed of a rigid material. Example rigid materials may include: metal alloys, plastics, ceramics, composites, glass, hybrid materials, etc. In one or more embodiments, the enclosure may be formed in one or more of the following approaches: injection molding, blow molding, rotational molding, thermoforming, three-dimensional (3D) printing. In injection molding, molten material is forced into a mold, then cooled to create a wide range of products, from small components to large housing parts. In blow molding, the plastic is extruded into a tube that's closed in a mold. The plastic is inflated like a balloon, pressed against the mold, and cooled. In rotational molding, a heated hollow mold is filled with melted material, which is then slowly rotated causing the material to disperse and stick to the walls of the mold. In thermoforming, a material sheet is heated until it becomes pliable, then formed to a specific shape on a mold. Once cooled, the material retains the molded shape. In one or more embodiments of 3D printing, a 3D printer performs stereolithography 3D printing. In one or more embodiments, the 3D printer uses a resin vat that is cured with light, e.g., provided by a projector or laser. The object is formed in the resin vat layer by layer as the light cures additional layers of resin onto the object. In other embodiments of 3D printing, a 3D printer layers molten material to form 3D objects according to a blueprint. In other embodiments of 3D printing, a 3D printer performs selective laser sintering (SLS) printing. In such embodiments, the 3D printer leverages a controllable laser to sinter thermoplastic powder on a powder bed, thereby forming the object from the sintered powder. In other embodiments, a 3D printer performs multi jet fusion (MJF) 3D printing. In such embodiments, the 3D printer applies fusing and detailing agents across a bed of nylon powder, which is then fused with heat (e.g., from thermal elements or light-emitting elements). The 3D printer iteratively builds layers to form the 3D printed objected. The 3D printed object can be cured, sanded, or otherwise post-processed. In one or more embodiments, each enclosure half 110 is monolithically formed. In other embodiments, portions of the enclosure halves 110 may be separately formed and connected together, e.g., via adhesive, welding, etc.

FIG. 2A illustrates an internal view of an enclosure half 200, according to one or more embodiments. FIG. 2B illustrates a perspective view of the enclosure half 200, according to one or more embodiments. The enclosure half 200 is an embodiment of the enclosure half 110 of the splash guard 100. The enclosure half 200 includes the side wall 210, the top wall 220, the bottom wall 230, the protrusion 240, and the arm 250. In other embodiments, the enclosure half 200 may include additional, fewer, or different components than those listed herein.

The enclosure is formed by the side walls 210, the top walls 220, the bottom walls 230, and the protrusions 240 of the two enclosure halves 200. The side wall 210 runs parallel to the center axis 205. The top wall 220 is perpendicular to the center axis 205 and connects to the side wall 210 at the top edge 212. The bottom wall 230 connects to the side wall 210 at the bottom edge 214. The protrusion 240 connects to an inner surface of the side wall 210.

In one or more embodiments, the bottom wall 230 forms a basin 232 (illustrated in FIG. 2B) to hold leaked liquid from the syringe assembly. The bottom wall 230 may have a curved shape. For example, the bottom wall 230 may include a V-shape (as shown in FIGS. 2A & 2B) radially distant from the center axis 205 that is rotated around the center axis 205 to form the curved shape of the bottom wall 230. An outer edge of the bottom wall 230 connects to the bottom edge 214 of the side wall 210. An inner edge of the bottom wall 230 forms an opening in the enclosure where a portion of the syringe assembly may extend out of (e.g., an orifice of the filter of the syringe assembly). An intermediate point 234 of the curved shape of the bottom wall 230 extends further from the bottom edge 214 of the side wall 210 than the outer edge and the inner edge of the bottom wall 230. The tapered nature of the bottom wall 230 aids in centering a vial with the bottom portion of the syringe assembly extending below and out of the enclosure. The bottom wall 230 may further include two face plates 236 coupled to lateral edges of the curved shape positioned along the bifurcation plane between the two enclosure halves 200. The basin 232 can hold liquid at a volume determined by the curved shape of the bottom wall. In one or more embodiments, the curved shape can be semi-circular, rectangular, U-shaped, etc. In one or more embodiments, a seal may be incorporated around the inner edge of the curved shape of the bottom wall 230. The inner edge may be sized to fit an orifice of the syringe assembly (e.g., an orifice of the filter).

The protrusion 240 aids in securing the position of the splash guard (e.g., the splash guard 100) relative to the syringe assembly, when engaged. The protrusion 240 divides the enclosure cavity into two chambers: a first chamber 242 and a second chamber 244. The first chamber 242 is configured to hold a top portion of the syringe assembly, e.g., inclusive of the syringe of the syringe assembly. The second chamber 244 is configured to hold a bottom portion of the syringe assembly, e.g., inclusive of the filter of the syringe assembly. The protrusion 240 is sized to create an opening between the two chambers of smaller dimension than the filter of the syringe assembly. Accordingly, when engaged in the optimal placement (an example shown in FIG. 3B), the bottom wall 230 and the protrusion 240 secure the filter in the second chamber 244. In other words, the filter can pass neither through the opening formed by the protrusion 240 nor the opening formed by the bottom wall 230. The dimensions of the second chamber 244 are sized to fit the filter of the syringe assembly. A width (as measured perpendicular to the center axis 205) is greater than the width of the filter. A height (as measured parallel to the center axis 205) is greater than the height of the filter. The second chamber 244 may be connected to the basin formed by the bottom wall 230. In one or more embodiments, the protrusion 240 is tapered in shape. An outer edge of the protrusion 240 may be thicker than an inner edge of the protrusion 240. The tapered thickness of the protrusion 240 funnels any liquid leaked into the first chamber 242 to drop into the second chamber 244, which may be further collected by the basin of the bottom wall 230. In one or more embodiments, the protrusion 240 can form its own basin, e.g., to hold liquid leaked into the first chamber 242. In one or more embodiments, a seal may be coupled to the inner edge of the protrusion 240.

The arm 250 includes portions that are angled relative to one another. For example, as shown in FIG. 2B, the arm includes a first portion 252 that extends from the hinge 260 towards a first end of the arm 250, i.e., which connects to the outer surface of the side wall of the enclosure. The second portion 254 extends from the hinge 260 towards the second end of the arm 250. The two portions are angled in a plane perpendicular to the hinge axis (i.e., the hinge axis being parallel to the center longitudinal axis). The angle a between the two portions dictates the maximum opening angle of the enclosure. Twice the supplementary angle to angle a provides the maximum opening angle of the enclosure. As the two arms 250 of the enclosure halves draw together (e.g., as force is applied by a user), the second portions 254 of the two arms 250 would be angularly offset by a minimal amount, e.g., thickness of the arm 250. As the second portions 254 draw together, the first portions 252 would rotate away from another (about the hinge 260) at an angle corresponding to how closely the second portions 254 of the two arms 250 are drawn together.

### Example Operation of the Splash Guard

FIG. 3A illustrates a first view of the splash guard 300 disengaged from a syringe assembly 310, according to one or more embodiments. FIG. 3B illustrates a second view of the splash guard 300 engaged to a syringe assembly 310, according to one or more embodiments. The splash guard 300 is an embodiment of the splash guard 100.

In FIG. 3A, the splash guard 300 is in equilibrium in the closed state. The walls of the enclosure are impelled together from the spring-loaded hinge, along the hinge axis. When in the closed state, the enclosure has two openings into the internal cavity-one opening at the top of the enclosure for fitting a top portion of the syringe assembly 310 and one opening at the bottom of the enclosure for fitting a bottom portion of the syringe assembly 310.

FIG. 3B illustrates the splash guard 300 in an opened state engaging with the syringe assembly 310. As a user applies an actuation force to the handle of the splash guard 300, the enclosure is opened to an opened state, wherein the splash guard 300 can clamp onto the syringe assembly 310. The user would position the splash guard 300 such that the filter of the syringe assembly 310 is positioned within the enclosure. In one or more embodiments, the splash guard 300 may include a protrusion dividing the enclosure cavity into a top chamber (i.e., a first chamber) and a bottom chamber (i.e., a second chamber). In such embodiments, the user may position the splash guard 300 such that the filter is positioned with the bottom chamber. In one or more embodiments, the syringe assembly 310 may include multiple filters coupled to the syringe in series. In such embodiments, the user may position the splash guard 300 such that the bottom filter (not the filter adjacent to the syringe) is positioned within the bottom chamber of the enclosure.

### Example Construction

In one or more embodiments, the splash guard may be manufactured with a three-dimensional printer. The enclosure halves of the splash guard may be printed together or separately. In some embodiments, the enclosure is printed separately from the handle, with the two subsequently joined together.

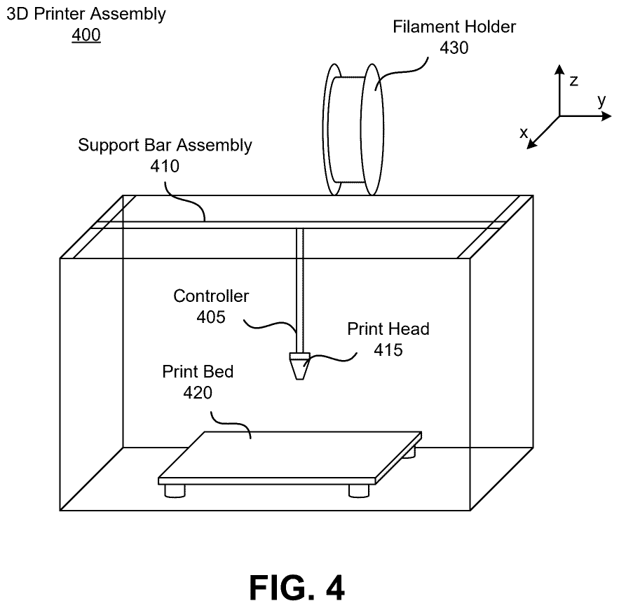

FIG. 4 illustrates an example 3D printer assembly 400, according to one or more embodiments. The 3D printer assembly 400 may include a print head 415 coupled to a controller 405 connected to a support bar assembly 410. Controller 405 may carry the print head 415 for printing a 3D object. Controller 405 may be movable in one or more directions, thus controlling the movements of print head 415. A print bed 420 provides a surface upon which a 3D object may be fabricated. A filament holder 430 holds the filament fed into the print head 415.

Controller 405 may be programmed to be moved along the three translational axes. The controller 405 is coupled to the support bar assembly 410. For example, the support bar assembly 410 may include a center support bar coupled to lateral support bars (parallel to the print bed 420) positioned on opposite sides of the 3D printer assembly 400. Sliding movement of the controller 405 along the center support bar provides control of the print head 415 along the y-axis. Sliding movement of the center support bar relative to the lateral support bars provides control of the print head 415 along the x-axis. The lateral support bars may further be coupled to vertical support bars (perpendicular to the print bed 420) disposed along the corners of the 3D printer assembly 400. Sliding movement of the lateral support bars relative to the vertical support bars provides control of the print head 415 along the z-axis. With the support bar assembly 410, the controller 405 can control three-dimensional translation of the print head 415 relative to the print bed 420. Alternatively, the print bed 420 may be movable in one or more of the three translational axes when needed to create a desired 3D object. In general, either the controller 405 or the print bed 420 may be movable in each one of the three axes in coordination to enable 3D printing of an object. Movements of controller 405 and print bed 420 may be directed a computer (not shown) with which the 3D printer assembly 400 is in communication (either wired or wirelessly). The computer may direct the movements through a digital model of the 3D object being printed. The digital model may be a 3D information file describing the 3D printable object in three dimensions.

To create a 3D object, the print head 415 may dispense droplets of a liquid-to-solid material such as plastic on the top surface of print bed 420. 3D objects are generally created in this manner layer by layer. Programmed movement of the print head 415 and/or the print bed 420 while the material is being dropped on the top surface of the print bed 420 may result in a first layer of the 3D object of a desired shape. Additional layers of the material may then be dropped layer by layer to eventually create a complete 3D object. In one or more embodiments, the print head 415 dispenses material from a filament fed into the print head 415 from the filament holder 430. For example, the filament may be spooled on the filament holder 430.

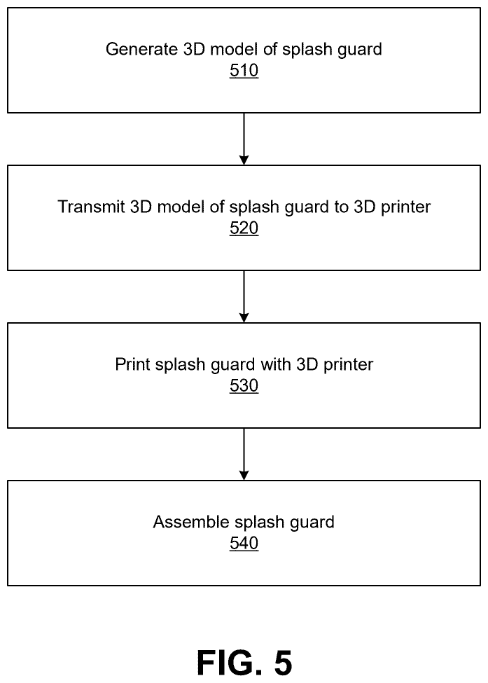

FIG. 5 illustrates a method flowchart for a process of manufacturing a splash guard (e.g., the splash guard 100) for a syringe assembly, according to one or more embodiments. Some or all of the steps may be automated by one or more machines.

The process includes generating 510 a 3D model of the splash guard. The 3D model may include spatial dimensions of the splash guard. The 3D model may further include printing support structures that aid in the 3D printing process. Such support structures may be removed post-3D-printing.

The process includes transmitting 520 the 3D model of the splash guard to a 3D printer (e.g., the 3D printer assembly 400 of FIG. 4). The 3D printer can determine the print instructions based on the 3D model. For example, the print instructions may include an orientation to print the splash guard, speed of the printing, movement of the print head, etc.

The process includes printing 530 the splash guard with the 3D printer. Based on the print instruction and/or the 3D model, the 3D printer dispenses liquid-to-solid material on a printing bed to craft the splash guard in a layer-by-layer printing process. Once printed, the splash guard may be cured, coated, sanded, or any other post-printing finishing process.

The process includes assembling 540 the splash guard. In some embodiments, the two enclosure halves of the splash guard may be 3D-printed separately. To join the two enclosure halves, a rod and/or spring is used to join the two enclosure halves. For example, the rod may be inserted into holes of the arms to join the two enclosure halves at the hinge. In other examples, a torsion spring is inserted into the holes of the arms with hinges of the torsion spring coupled lengthwise to the arms. In some embodiments, the splash guard may be further subdivided into constituent parts. In such embodiments, each constituent part may be separately printed. Assembly may include adhering the parts together, e.g., with welding, glue, etc.

### Additional Considerations

The foregoing description of the embodiments has been presented for the purpose of illustration; many modifications and variations are possible while remaining within the principles and teachings of the above description.

Any of the steps, operations, or processes described herein may be performed or implemented with one or more hardware or software modules, alone or in combination with other devices. In some embodiments, a software module is implemented with a computer program product comprising one or more computer-readable media storing computer program code or instructions, which can be executed by a computer processor for performing any or all of the steps, operations, or processes described. In some embodiments, a computer-readable medium comprises one or more computer-readable media that, individually or together, comprise instructions that, when executed by one or more processors, cause the one or more processors to perform, individually or together, the steps of the instructions stored on the one or more computer-readable media. Similarly, a processor may comprise one or more subprocessing units that, individually or together, perform the steps of instructions stored on a computer-readable medium.

Embodiments may also relate to a product that is produced by a computing process described herein. Such a product may store information resulting from a computing process, where the information is stored on a non-transitory, tangible computer-readable medium and may include any embodiment of a computer program product or other data combination described herein.

Where values are described as “approximate” or “substantially” (or their derivatives), such values should be construed as accurate +/−10% unless another meaning is apparent from the context. From example, “approximately ten” should be understood to mean “in a range from nine to eleven.”

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive “or” and not to an exclusive “or”. For example, a condition “A or B” is satisfied by any one of the following: A is true (or present) and B is false (or not present); A is false (or not present) and B is true (or present); and both A and B are true (or present). Similarly, a condition “A, B, or C” is satisfied by any combination of A, B, and C being true (or present). As a not-limiting example, the condition “A, B, or C” is satisfied when A and B are true (or present) and C is false (or not present). Similarly, as another not-limiting example, the condition “A, B, or C” is satisfied when A is true (or present) and B and C are false (or not present).

The language used in the specification has been principally selected for readability and instructional purposes, and it may not have been selected to narrow the inventive subject matter. It is therefore intended that the scope of the patent rights be limited not by this detailed description, but rather by any claims that issue.

What is claimed is:

1. A splash guard for a syringe assembly, the splash guard comprising:
first and second enclosure halves, each enclosure half comprising:
a body having a side wall, a top wall connected to the side wall along a top edge of the side wall, and a bottom wall connected to the side wall along a bottom edge of the side wall;
an internal protrusion connected to an inner surface of the side wall and structured to guide the syringe assembly to a particular placement within the enclosure; and
an arm comprising a first end connected to an outer surface of the side wall opposite the inner surface, and a second end opposite the first end for gripping by a user; and
a hinge, pivotably coupling the enclosure halves, biasing the arms towards an equilibrium state in which the second ends of the arms are positioned away from one another and the first ends of the arms impel the two enclosure halves together such that the two enclosure halves form a substantially enclosed cavity when engaged with the syringe assembly.

2. The splash guard of claim 1, wherein the body of each enclosure half is of semi-cylindrical shape, such that the enclosure is of cylindrical shape.

3. The splash guard of claim 1, wherein the top wall of each enclosure half includes a cutaway portion, such that, in the equilibrium state, the top walls of the two enclosure halves form an opening to the enclosure for placement of a syringe of the syringe assembly.

4. The splash guard of claim 3, wherein, for each enclosure half, the top wall is planar and perpendicular to the side wall.

5. The splash guard of claim 1, wherein the bottom wall of each enclosure half includes a cutaway portion, such that, in the equilibrium state, the bottom walls of the two enclosure halves form an opening to the enclosure for placement of a filter of the syringe assembly.

6. The splash guard of claim 5, wherein, for each enclosure half, the bottom wall is planar and perpendicular to the side wall.

7. The splash guard of claim 5, wherein each enclosure half further comprises:
a seal coupled to an inner edge of the cutaway portion of the bottom wall to form a waterproof seal against a filter of the syringe assembly.

8. The splash guard of claim 1, wherein the protrusion of each enclosure half is of semi-annular shape, such that the protrusions form an annular shape.

9. The splash guard of claim 1, wherein each enclosure half further comprises:
a seal coupled to an inner edge of the protrusion to form a seal against the syringe assembly.

10. The splash guard of claim 1, wherein the internal protrusions of the two enclosure halves divide the enclosure into a first chamber and a second chamber,
wherein the first chamber is formed by the internal protrusions, the side wall, and the top wall and configured to hold at least a portion of the syringe of the syringe assembly, and
wherein the second chamber is formed by the internal protrusions, the side wall, and the bottom wall and configured to enclose the filter of the syringe assembly.

11. The splash guard of claim 10, wherein, for each enclosure half, the bottom wall is curved with an intermediate point of the bottom wall extending further away from a plane formed by the bottom edge of the side wall than edges of the bottom wall.

12. The splash guard of claim 1, wherein the hinge comprises a torsion spring.

13. The splash guard of claim 12, wherein each enclosure half is formed through a three-dimensional printing process.

14. The splash guard of claim 1, wherein the enclosure halves are formed from a material that is at least partially transparent.

15. A method for manufacturing a splash guard for a syringe assembly, the method comprising:
transmitting a three-dimensional (3D) model for the splash guard to be printed on a 3D printer, the 3D model for the splash guard comprising:
first and second enclosure halves, each enclosure half comprising:
a body having a side wall, a top wall connected to the side wall along a top edge of the side wall, and a bottom wall connected to the side wall along a bottom edge of the side wall,
an internal protrusion connected to an inner surface of the side wall and structured to guide the syringe assembly to a particular placement within the enclosure, and
an arm comprising a first end connected to an outer surface of the side wall opposite the inner surface, and a second end opposite the first end for gripping by a user;
fabricating the enclosure halves using the 3D printer; and
joining the enclosure halves at a pivot with a hinge biasing the arms towards an equilibrium state in which the second ends of the arms are positioned away from one another and the first ends of the arms impel the two enclosure halves together such that the two enclosure halves form a substantially enclosed cavity when engaged with the syringe assembly.

16. The method of claim 15, wherein each enclosure half further comprises:
a seal coupled to an inner edge of the cutaway portion of the bottom wall to form a waterproof seal against a filter of the syringe assembly.

17. The method of claim 15, wherein each enclosure half further comprises:
a seal coupled to an inner edge of the protrusion to form a seal against the syringe assembly.

18. The method of claim 15, wherein the internal protrusions of the two enclosure halves divide the enclosure into a first chamber and a second chamber,
wherein the first chamber is formed by the internal protrusions, the side wall, and the top wall and configured to hold at least a portion of the syringe of the syringe assembly, and
wherein the second chamber is formed by the internal protrusions, the side wall, and the bottom wall and configured to enclose the filter of the syringe assembly.

19. The method of claim 15, wherein fabricating the enclosure halves using the 3D printer comprises printing each enclosure half monolithically.

20. The method of claim 15, wherein fabricating the enclosure halves using the 3D printer comprises printing the enclosure halves with a material that is at least partially transparent.
